SEQUENCE LISTING (15 sequences)

SEQ ID NO: 1 - DNA, 22 bp, Artificial Sequence
  Primer P1
gccctttactctaaagttgcgg

SEQ ID NO: 2 - DNA, 32 bp, Artificial Sequence
  Primer P2
gtctacagacatggatgagcgatgatgatatc

SEQ ID NO: 3 - DNA, 40 bp, Artificial Sequence
  Primer P3
catcgctcatccatgtctgtagacaaattgtgaaaggatg

SEQ ID NO: 4 - DNA, 44 bp, Artificial Sequence
  Primer P4
cttcgctctcactgagctccactaatattaataaactatcgaag

SEQ ID NO: 5 - DNA, 30 bp, Artificial Sequence
  Primer P5
ggagctcagtgagagcgaagcgaacacttg

SEQ ID NO: 6 - DNA, 42 bp, Artificial Sequence
  Primer P6
gcggtacctgatctagatctcgggttcttcaaatatttctcc

SEQ ID NO: 7 - DNA, 46 bp, Artificial Sequence
  Primer P7
cccgagatctagatcaggtaccgcaacgaattcggccgaaaagtgc

SEQ ID NO: 8 - DNA, 50 bp, Artificial Sequence
  Primer P8
cacttccctcctcatttttaacgcgtagctgctgcgaagacaactaaaac

SEQ ID NO: 9 - DNA, 51 bp, Artificial Sequence
  Primer P9
cgcgttaaaaatgaggagggaagtgttatggatgtggaggttaagaaaaac

SEQ ID NO: 10 - DNA, 37 bp, Artificial Sequence
  Primer P10
ccctttagtgtcaattcactagccatccgtatgatcc

SEQ ID NO: 11 - DNA, 30 bp, Artificial Sequence
  Primer P11
ggctagtgaattgacactaaagggatccag

SEQ ID NO: 12 - DNA, 31 bp, Artificial Sequence
  Primer P12
cgcggccgcggatcagccctttacgctcgat

SEQ ID NO: 13 - DNA, 42 bp, Artificial Sequence
  Primer P13
agggctgatccgcggccgcgcgtcaacaatgacctttatgcc

SEQ ID NO: 14 - DNA, 23 bp, Artificial Sequence
  Primer P14
cagaggggatggatcgaaatatg

SEQ ID NO: 15 - DNA, 5776 bp, Artificial Sequence
  DNA sequence of the amyE::specR Ppst sigF insert
ctcaggtctggaaaagaaaagaggcgtactgcctgaacgagaagctatcaccgcccagcctaaacggatatcatcatcgctcatccatgtctgtagacaagagtccagaccttttcttttctccgcatgacggacttgctcttcgatagtggcgggtcggatttgcctatagtagtagcgagtaggtacagacatctgttattgtgaaaggatgtacttaaacgctaacggtcagctttattgaacagtaatttaagtatatgtccaatctagggtaagtaaattgagtatcaatataaataacactttcctacatgaatttgcgattgccagtcgaaataacttgtcattaaattcatatacaggttagatcccattcatttaactcatagttatatttctttatatgaacataatcaacgaggtgaaatcatgagcaatttgattaacggaaaaataccaaatcaagcgattcaaacattaaaaatcgtaaaagatttgaaatatacttgtattagttgctccactttagtactcgttaaactaattgcctttttatggtttagttcgctaagtttgtaatttttagcattttctaaatcaatagttggagtatatctatttggttcagcagtaaatggtggtttacgcattaacagcgatgtagatgttctagtcgtcgtgaatcattaaaccttcaagttatcaacctcatatagataaaccaagtcgtcatttaccaccaaatgcgtaattgtcgctacatctacaagatcagcagcacttagtaagtttacctcaattaactcgaaaaaaactaacagaaagactaatgactatatcaggaaagattggaaataggattctgttagaccacttgaagttacggtcaaatggagttaattgagctttttttgattgtctttctgattactgatatagtcctttctaacctttatgcctaagacaatctggtgaacttcaatgccttataaataggagtgaagttgtcccttggcaatatcctccaaaaagagaatttatatacggtgagtggctcaggggtgaattgagaatggacaaattcaaatatttatcctcacttcaacagggaaccgttataggaggtttttctcttaaatatatgccactcaccgagtccccacttaaactcttacctgtttaagtggaaccaagctatgatcctgatttggctattgttttagcacaagcaagaaagaatagtatttctctatttggtcctgattttcaagtatacttgtctccccttggttcgatactaggactaaaccgataacaaaatcgtgttcgttctttcttatcataaagagataaaccaggactaagaagttcatatgaacagagggtacctttgacagatattcgaagagcaattaaggattctttgccagaactaattgaggggataaaaggtgatgagcgtaatgtaattttaccctagctccatggaaactgtctataagcttctcgttaattcctaagaaacggtcttgattaactcccctattttccactactcgcattacattaaaattgggatcgaggaatgtggcaaacagtgactactggtgaaattacctcgaaagatgtcgctgcagaatgggctatacctcttttacctaaagagcatgtaatttactggacttacaccgtttgtcactgatgaccactttaatggagctttctacagcgacgtcttacccgatatggagaaaatggatttctcgtacattgaaatgaccttatagctagaaaaggctatcggggagagtgtgatgataagtgggaaggactatattcaaaggtgaaagcactcgttaagtatatgaaaaattctatagaaatatcgatcttttccgatagcccctctcacactactattcacccttcctgatataagtttccactttcgtgagcaattcatatactttttaagatatcttacttctctcaattaggctaattttattgcaataacaggtgcttacttttaaaactactgatttattgataaatattgaacaatttttgggaagaataaagtgaagagagttaatccgattaaaataacgttattgtccacgaatgaaaattttgatgactaaataactatttataacttgttaaaaacccttcttatttccgtcctcttgtgaaattagagaacgctttattactttaatttagtgaaacaatttgtaactattgaaaatagaaagaaattgttccttcgtagtttattgcaggagaacactttaatctcttgcgaaataatgaaattaaatcactttgttaaacattgataacttttatctttctttaacaaggaagctatcaaataaaatattagtggagctcagtgagagcgaagcgaacacttgattttttaattttctatcttttataggtcattagagtatacttatttgtccataaactatttataatcacctcgagtcactctcgcttcgcttgtgaactaaaaaattaaaagatagaaaatatccagtaatctcatatgaataaacaggatatttgatattagcagcataatagatttattgaataggtcatttaagttgagcatattagaggaggaaaatcttggagaaatatttgaagaacccgagactagatcagaatcgtcgtattatctaaataacttatccagtaaattcaactcgtataatctcctccttttagaacctctttataaacttcttgggctctagatctagtcgtaccgcaacgaattcggccgaaaagtgcttgataagtattttgaactgaaatcaaaacaatcaaaagaaaatactgtcgaaaaaaataagacaaaatccatggcgttgcttaagccggcttttcacgaactattcataaaacttgactttagttttgttagttttcttttatgacagctttttttatttctgttttaggaaaaagaagctacagaataggactttttcattaaaacacctgcattgaaccatgcaggtgtttttttgtgcgcccgttcttttcgtgcgattctatacctttttcttcgatgtcttatcctgaaaaagtaattttgtggacgtaacttggtacgtccacaaaaaaacacgcgggcaagaaaagcacgcgtaagatatgaaagttcaagcttaatttacaaaaccttaataattcatttaaacaagattaatactgaaaagttacgctatacccgtaggacaaattacacatttacgcgtttcaagttcgaattaaatgttttggaattattaagtaaatttgttctaattatgacttttcaatgcgatatgggcatcctgtttaatgtgtaaatgcgcggaggattaagaagtgaaaccatttaaaaaaatcacactgatgttcatcatgtccgttttagttgtcttcgcagcagctacgcgttaaaaatgaggagggcctcctaattcttcactttggtaaatttttttagtgtgactacaagtagtacaggcaaaatcaacagaagcgtcgtcgatgcgcaatttttactcctcccaagtgttatggatgtggaggttaagaaaaacggcaaaaacgctcagctgaaggatcatgaagtaaaggaattaatcaaacaagccaaaatggcgaccagttcacaatacctacacctccaattctttttgccgtttttgcgagtcgacttcctagtacttcatttccttaattagtttgtttcggttttaccgctggtccaggcaagagacctcctcatagaaaaaaacatgcgtcttgtttggtctgtcgtacagcggtttttaaacagaggatatgagcctgacgatctcttccagagtccgttctctggaggagtatctttttttgtacgcagaacaaaccagacagcatgtcgccaaaaatttgtctcctatactcggactgctagagaaggtcttcggctgcatcgggctgttaaaatctgttgacaaatttgatttaacctatgatgtgcgtttttcaacgtatgcagtgccgatgattatcggagaaatccaagccgacgtagcccgacaattttagacaactgtttaaactaaattggatactacacgcaaaaagttgcatacgtcacggctactaatagcctctttaggtacgatttatccgtgatgacggaaccgtaaaggtatcacggtcattaaaagagcttggaaacaaaatccggcgcgcgaaggatgagctttcgaaaacactgtgctaaataggcactactgccttggcatttccatagtgccagtaattttctcgaacctttgttttaggccgcgcgcttcctactcgaaagcttttgtgacggcagagtgccgacggtgcaggagatcgctgaccatttggagattgaagctgaggatgttgtactggcccaagaggcggtaagggctccatcttcgattcccgtctcacggctgccacgtcctctagcgactggtaaacctctaacttcgactcctacaacatgaccgggttctccgccattcccgaggtagaagctaagacgaaaccgtttatgaaaatgacggagatccgattaccctgcttgatcaaatcgctgacaactcagaagaaaaatggtttgacaaaattgcgctgaaagatgctttggcaaatacttttactgcctctaggctaatgggacgaactagtttagcgactgttgagtcttctttttaccaaactgttttaacgcgactttctagcgatcagcgatttggaggaaagggaaaaactaatcgtctatctcagatattataaagaccagacacagtccgaggtggtgagcggctcgggatctcttcgctagtcgctaaacctcctttccctttttgattagcagatagagtctataatatttctggtctgtgtcaggctccaccgactcgccgagccctagagacaggtgcaggtttccaggcttgaaaagaaaatattaaaacagatcaaggttcaaatggatcatacggatggctagtgaattgacactaaagggatccagagtccacgtccaaaggtccgaacttttcttttataattttgtctagttccaagtttacctagtatgcctaccgatcacttaactgtgatttccctaggtctagcggcaacacgctaatcaataaaaaaacgctgtgcggttaaagggcacagcgtttttttgtgtatgaatcgaaaaagagacagatcgcaggtctcaaatcgccgttgtgcgattagttatttttttgcgacacgccaatttcccgtgtcgcaaaaaaacacatacttagctttttctcttgtctagcgtccagagtttaatcgagcgtaaagggctgatccgcggccgcgcgtcaacaatgacctttatgccatattcttcagcggctgcacacattttttaaattcttgttcagtattagctcgcatttcccgactaggcgccggcgcgcagttgttactggaaatacggtataagaagtcgccgacgtgtgtaaagaaatttaagaacaagtcatcctaagtaacggttgccaatttgatacgatgtcggctgatacagccagtaccagttcgacatgcttttatctccttgattcccttcctttacttggttaaggattcattgccaacggttaaactatgctacagccgactatgtcggtcatggtcaagctgtacgaaaatagaggaactaagggaaggaaatgaaccaatt

(19) United States
(12) Patent Application Publication
(10) Pub. No.: US 2013/0224757 A1
(43) Pub. Date: Aug. 29, 2013

(54) INDUCED SPORULATION SCREENING METHOD

(71) Applicants: Mads Bjoernvad, Virum (DK); Poul Erik Pedersen, Farum (DK); Marco Malten, Copenhagen NV (DK)

(72) Inventors: Mads Bjoernvad, Virum (DK); Poul Erik Pedersen, Farum (DK); Marco Malten, Copenhagen NV (DK)

(73) Assignee: NOVOZYMES A/S, Bagsvaerd (DK)

(21) Appl. No.: 13/816,891

(22) PCT Filed: Aug. 18, 2011
(86) PCT No.: PCT/EP11/64200

§ 371 Date: May 16, 2013

(30) Foreign Application Priority Data
Aug. 19, 2010 (EP) 10173360.8

(51) Int. Cl. C12Q 1/68

(52) U.S. Cl. CPC C12Q 1/6897
USPC 435/6.18; 435/320.1; 435/252.31

(57) ABSTRACT

The present invention relates to a method of screening for an improved enzyme variant, said method comprising the steps of: (i) providing a recombinant host cell capable of sporulating comprising a polynucleotide encoding a sporulation factor and a polynucleotide encoding an enzyme variant which are operably linked to an inducible promoter; (ii) culturing the host cell under conditions suitable to induce the formation of spores; (iii) culturing the spores obtained in step (ii) in a medium containing a substrate for the enzyme variant; and (iv) determining the activity of the enzyme variant.

### SEQUENCE LISTING

The present invention comprises a sequence listing.

### FIELD OF THE INVENTION

The present invention relates to a method for screening enzymes using the recombinant cell in which sporulation is inducible, e.g., by phosphorous starvation. The invention also relates to tools for carrying out said method, incl. the recombinant cell and polynucleotide constructs.

### BACKGROUND OF THE INVENTION

Enzymes which can stand extreme conditions are highly desirable for use in industrial applications. In many protein screening projects, the actual enzyme screening conditions do not allow growth of the host strains. Also, multiple times of transfer of the host cell from one medium to another during screening can result in contamination and difficulty with automation. A positive screening system which effectively allows selection of improved enzymes under conditions inhibiting growth is thus very desirable.

A spore is a reproductive structure that is adapted for dispersal and survival for extended periods of time in unfavorable conditions. Once conditions are favorable, the spore can develop into a new organism. Spores form part of the life cycles of many organisms, such as bacteria, plants, algae and fungi. A sporulating bacterium, especially Bacillus, is an ideal system not only for investigating the distinctive regulation of gene expression but also for selectively expressing polypeptides.

Generally, there are six stages during sporulation, i.e., stages 0-VI. Several hundreds of genes were identified to participate in sporulation, and during each stage, there are different regulatory genes (EP1391502). For instance, sigF, also called spollAC, is a gene encoding an essential sporulation factor during stage II of sporulation. Traditionally, sporulation-associated genes were mutated or deleted so that the process of sporulation was inhibited or inactivated for improved expression of polypeptides (EP1391502).

### SUMMARY OF THE INVENTION

The present invention provides a screening system capable of effectively screening enzymes under extreme conditions, such as high temperature, low nutrients, presence of toxins or detergents etc.

We describe the introduction of a stress inducible promoter operably linked to a gene encoding a sporulation factor into a host cell. The recombinant cell may conveniently be used to screen an enzyme under extreme conditions by selecting clearing zones in a selective medium containing a substrate of the enzyme. The invention is applicable both in library screening and industrial applications such as detergents.

In a first aspect, the invention relates to a method of screening for an improved enzyme variant, said method comprising the steps of:
(i) providing a recombinant host cell capable of sporulating comprising a polynucleotide encoding a sporulation factor and a polynucleotide encoding an enzyme variant which are operably linked to an inducible promoter;
(ii) culturing the host cell under conditions suitable to induce the formation of spores;
(iii) culturing the spores obtained in step (ii) in a medium containing a substrate for the enzyme variant; and
(iv) determining the activity of the enzyme variant.

In a second aspect, the invention relates to a nucleic acid construct comprising a polynucleotide encoding a sporulation factor and a polynucleotide encoding an enzyme variant which are operably linked to an inducible promoter.

In a third aspect, the invention relates to a recombinant expression vector comprising the nucleic acid construct of the second aspect.

A final aspect of the invention relates to a recombinant cell comprising the nucleic acid construct of the second aspect or the recombinant expression vector of the third aspect.

### BRIEF DESCRIPTION OF DRAWINGS

FIG. 1 shows the schematic amyE::spec^{R} Ppst sigF PCR fragment obtained in Example 1.

### DEFINITIONS

Prior to a discussion of the detailed embodiments of the invention, a definition of specific terms related to the main aspects of the invention is provided.

In accordance with the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y.: DNA Cloning: A Practical Approach, Volumes I and II/D.N. Glover ed. 1985); Oligonucleotide Synthesis (M.J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

A “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary or quaternary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

A “gene” refers a nucleic acid sequence encoding a peptide, a polypeptide or a protein. In a particular embodiment the term “reporter gene” refers to a nucleic acid sequence encoding a reporter protein.

A “nucleic acid construct” is a nucleic acid molecule, either single- or double-stranded, which is isolated from a naturally occurring gene or which has been modified to contain segments of nucleic acid combined and juxtaposed in a manner that would not otherwise exist in nature. The term “nucleic acid construct” is synonymous with the term “expression cassette” when the nucleic acid construct contains all the control sequences required for expression of a coding sequence of the present invention. The term “coding sequence” is defined herein as a nucleic acid sequence that directly specifies the amino acid sequence of its protein product. The boundaries of the coding sequence are generally determined by a ribosome binding site (prokaryotes) or by the ATG start codon (eukaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

The term “promoter” is used herein for its art-recognized meaning to denote a sequence flanking the gene containing DNA sequences that provide for the binding of RNA polymerase and initiation of transcription and furthermore it contains DNA sequences that are responsible for the regulation of the transcription of the gene. Promoter sequences are commonly, but not always, found in the 5′ non-coding regions of genes. In a particular embodiment of the invention the promoter is an inducible promoter, e.g. a stress induced promoter, and specifically, a low-phosphate inducible promoter (also called phosphorus starvation inducible promoter, such as, Ppst).

The term “inducible promoter” is used herein as a promoter whose activity is induced by the presence or absence of biotic or abiotic factors. Inducible promoters are a very powerful tool in genetic engineering because the expression of genes operably linked to them can be turned on or off at certain stages of development of an organism or in a particular tissue.

Examples of suitable inducible promoters include, but are not limited to, the low phosphate or phosphorous starvation inducible promoter, pstS; the tetracyclin inducible promoter (Geissendörfer M, Hillen W, 1990, Regulated expression of heterologous genes in Bacillus subtilis using the Tn10 encoded tet regulatory elements. Appl Microbiol Biotechnol 33:657-663); the xylose inducible promoters, such as, PxyIA with XyIR as repressor (Kim L, Mogk A, Schumann W, 1996, A xylose-inducible Bacillus subtilis interation vector and its application. Gene 181:71-76); or the IPTG-inducible Spac promoter.

“Operably linked”, when referring to DNA segments, indicates that the segments are arranged so that they function in concert for their intended purposes, e.g. transcription initiates in the promoter and proceeds through the coding segment to the terminator.

A coding sequence is “under the control” of transcriptional and translational control sequences in a cell when RNA polymerase transcribes the coding sequence into mRNA, which is then trans-RNA spliced and translated into the protein encoded by the coding sequence.

“Heterologous” DNA refers to DNA not naturally located in the cell, or in a chromosomal site of the cell. Preferably, the heterologous DNA includes a gene foreign to the cell.

A cell has been “transfected” by exogenous or heterologous DNA when such DNA has been introduced inside the cell. A cell has been “transformed” by exogenous or heterologous DNA when the transfected DNA effects a phenotypic change.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A “polymerase chain reaction (PCR)” is a technique to amplify a single or few copies of a piece of DNA across several orders of magnitude, generating thousands to millions of copies of a particular DNA sequence. The method relies on thermal cycling, consisting of cycles of repeated heating and cooling of the reaction for DNA melting and enzymatic replication of the DNA. As PCR progresses, the DNA generated is itself used as a template for replication, setting in motion a chain reaction in which the DNA template is exponentially amplified. PCR can be extensively modified to perform a wide array of genetic manipulations.

A “primer” is a strand of nucleic acid that serves as a starting point for DNA replication. Primers containing sequences complementary to the target region along with a DNA polymerase are key components to enable selective and repeated amplification. In the present invention, a nucleic acid construct was constructed by PCR amplification and joined together via overlapping DNA primers.

In a preferred embodiment, the nucleic acid construct of the second aspect also comprises a polynucleotide encoding an antibiotic resistance selectable marker.

An “antibiotic” is a substance produced by fungi or bacteria which inhibit the growth of other microorganisms. Examples of antibiotics include ampicillin, kanamycin, tetracycline, chloramphenicol, neomycin and spectinomycin.

The term “low phosphate” means the level of phosphate in a medium is significantly lower than the normal level required for growth. The level of “low phosphate” depends on individual organisms. In a specific embodiment of the present invention, the level is lower than half of the normal level, e.g., in a working example, the cells were grown on more than 2-fold diluted, preferably more than 5-fold diluted Schaeffers agar in plates.

### DETAILED DESCRIPTION OF THE INVENTION

In its first aspect, the present invention relates to a method of screening for an improved enzyme variant, said method comprising the steps of:
(i) providing a recombinant host cell capable of sporulating comprising a polynucleotide encoding a sporulation factor and a polynucleotide encoding an enzyme variant which are operably linked to an inducible promoter;
(ii) culturing the host cell under conditions suitable to induce the formation of spores;
(iii) culturing the spores obtained in step (ii) in a medium containing a substrate for the enzyme variant; and
(iv) determining the activity of the enzyme variant.

In a particular embodiment, the enzyme obtained in step (iii) is selected by transferring the spores from step (ii) to the medium in step (iii) separately. In a preferred particular embodiment, the enzyme activity is determined by an agar overlay assay, more specifically, the spores in step (ii) are overlaid with the medium of step (iii) as described in detail below.

In a preferred aspect, the enzyme is an oxidoreductase, transferase, hydrolase, lyase, isomerase, or ligase. In a more preferred aspect, the enzyme is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, another lipase, mannosidase, mutanase, oxidase, pectinolytic enzyme, peroxidase, phytase, polyphenoloxidase, proteolytic enzyme, ribonuclease, transglutaminase or xylanase. In an even more preferred aspect, the enzyme is amylase. In another even more preferred aspect, the enzyme is protease.

### Cultivation of Cells

In the present invention, the cells are cultivated in a nutrient medium suitable for sporulation using methods well known in the art. For example, the cells may be cultivated by shake flask cultivation, and small-scale or large-scale fermentation (including continuous, batch, fed-batch, or solid state fermentations) in laboratory or industrial fermentors performed in a suitable medium and under conditions allowing the cells to sporulate. Suitable media are available from commercial suppliers or may be prepared according to published compositions (e.g., in catalogues of the American Type Culture Collection). The first medium in step (ii) may be any medium inducing sporulation, specifically, the medium may be single, chemically defined sporulation medium for Bacillus Subtilis (J. H. Hageman et al., 1984), chemically defined sporulation medium for Bacillus Subtilis (Leitch and Collier, 1996), or Schaeffers sporulation medium (SSM). In a preferred embodiment of the present invention, the medium is SSM. SSM is a commonly used general purpose sporulation medium for Bacillus subtilis and related species.

In a preferred embodiment of the present invention, the inducible promoter is the low phosphate or phosphorous starvation inducible promoter, pstS. To induce sporulation by low phosphate in the medium, the medium is at least 2× diluted, preferably at least 5× diluted.

In yet another preferred embodiment, the inducible promoter is the tetracyclin inducible promoter (Geissendörfer M, Hillen W, 1990, regulated expression of heterologous genes in Bacillus subtilis using the Tn10 encoded tet regulatory elements. Appl Microbiol Biotechnol 33:657-663); the xylose inducible PxyIA promoter with XyIR as repressor (Kim L, Mogk A, Schumann W, 1996, a xylose-inducible Bacillus subtilis interation vector and its application. Gene 181:71-76) or the isopropyl-beta-D-thiogalactopyranoside (IPTG)-inducible Spac promoter.

The screening of enzymes or the culturing of the host cell spores in the screening method may be performed under a stress condition, such as, high temperature, acidic pH, lack of nutrients, presence of one or more toxin and/or presence of one or more detergent.

In a preferred aspect, the culturing or screening may be conducted under a temperature of at least 70° C., 75° C., 80° C., 85° C., 90° C., and/or at a pH value of at most 6.0, 5.8, 5.6, 5.4, 5.2, 5.0, 4.8, 4.6, 4.4, 4.2, 4.0, 3.8, 3.6, 3.4, 3.2 or 3.0. In addition, the culturing or screening may be carried out in the presence of one or more detergent.

The enzymes of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

### Agar Overlay Assay

In the present invention first the cells are grown on plates with a solid growth medium. During the growth the cells produce the enzyme that shall be tested and also some cells sporulate. Then a medium for a top layer is prepared that contains a solidification agent and an enzyme substrate that can be used to detect the enzyme activity in the agar layer. The top agar can also contain a buffer system to control pH, inhibitors, salts to adjust the ionic strength, antibiotics to suppress contamination.

For solidification, agar is used in the present invention, which solubilises above 85° C. and solidifies around 32-40° C. It is autoclaved, kept at 60° C. and mixed with the other 60° C. warm ingredients of the top layer. Then a defined amount is poured onto the plate with solid growth medium and the grown cells. These plates can then be incubated at the desired conditions where the enzymatic reaction can be seen by a degradation of the enzyme substance either by a formation of a hallow, a shift in colour or fluorescence, a released fluoro or chromophore or other detectable changes. When the enzymatic activity allows the selection of the improved variant, the colony can be picked from below the enzymatic clearing zone. Due to the spore formation, the cells survived the screening conditions. These spores can be added to a new growth medium allowing germination and growth of the specific variant.

Other types of polymers or gelatinous substances can be used for solidification. This can be substance that melt at higher temperature and are solid at the wished incubation conditions, e.g. agarose, other solidifying carbohydrates, solidifying proteins such as gelatin, solidifying fats, solidifying inorganics such as silicates and silica gels. Solidification can also be obtained by polymerizing chemicals, e.g. polyacrylamide.

The present invention relates to a nucleic acid construct comprising a polynucleotide encoding a sporulation factor operably linked to an inducible promoter.

### Inducible Promoters.

In the context of the present invention stress inducible promoter, inducible promoter and inducible promoter gene are used as synonymous.

In the preferred embodiment of the present invention, the inducible promoter is a low-phosphate or phosphate-starvation inducible promoter. Phosphorus is an important element for the growth and metabolic process of many organisms, such as plants, bacteria. Chinese Patent ZL 200610075838.2 reported a phosphate-starvation induced promoter in Arabidopsis thaliana. There were also studies concerning the phosphate-starvation regulation in Bacillus (e.g., Le Thi Hoi et al., The phosphate-starvation response of Bacillus licheniformis. Proteomics, 2006, Vol (6), pp. 3582-3601; Ying Qi et al., The pst operon of bacillus subtilis has a phosphate-regulated promoter and is involved in phosphate transport but not in regulation of the pho regulon. Journal of Bacteriology. April 1997, pp. 2534-2539). Especially, Le Thi Hoi et al.(2006, supra) explored the phosphate-starvation stimulon of Bacillus Licheniformis at the transcriptional and translational level. Bacillus licheniformis has been used in industrial fermentation processes for many years. The genome sequence of Bacillus licheniformis DSM13 was published in “The complete genome sequence of Bacillus licheniformis DSM13, an organism with great industrial potential” (Veith B., J. Mol. Microbiol. Biotechnol. 2004, 7(4), pp. 204-211). Through the study of Le Thi Hoi et al., it was found that pstS gene was most strongly induced during phosphate starvation, even over 80-fold. Further, the sequence of the pstS gene was identified (Veith B., 2004, supra). Hence in a preferred embodiment of the present invention, the low-phosphate inducible promoter used herein was pstS from Bacillus licheniformis DSM13.

### Polynucleotide (Nucleic Acid) Sequence.

In the method of the present invention a polynucleotide sequence of interest may be obtained in various ways known in the art. Non-limiting examples are: isolation of wild type genes, generation of protein engineered variants, site directed mutagenesis, library screening.

As used herein the term “nucleic acid sequence” is intended to indicate any nucleic acid molecule of cDNA, genomic DNA, synthetic DNA or RNA origin. The term “sequence” is intended to indicate a nucleic acid segment which may be single- or double-stranded, and which may be based on a complete or partial nucleotide sequence encoding a polypeptide.

The nucleic acid sequence of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., 1989).

The nucleic acid sequence may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid sequence may be of non cult type, mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid sequence may also be prepared by polymerase chain reaction using specific primers, for instance as described in U.S. Pat. No. 4,683,202 or Saiki et al., Science 239 (1988), 487-491.

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See e.g. Innis et al., 1990, A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain producing the polypeptide, or from another related organism and thus, for example, may be an allelic or species variant of the polypeptide encoding region of the nucleic acid sequence.

The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

### Polynucleotide Encoding a Sporulation Factor

Generally, there are six stages during sporulation, i.e., stages 0-VI. Several hundreds of genes were identified to participate in sporulation, and during each stage, there are different regulatory genes (EP1391502). For instance, sigF, also called spoIIAC, is a gene encoding an essential sporulation factor during stage II of sporulation.

In one embodiment, the sporulation factor is one that takes part in stages II-III of sporulation; more preferably, SpoIIAC, SpoIIE, SpoIIGB, and SpoIISB; most preferably SpoIIAC.

### Nucleic Acid Sequence Library

Preparation of a nucleic acid sequence library can be achieved by use of known methods.

Procedures for extracting genes from a cellular nucleotide source and preparing a gene library are described in e.g. Pitcher et al., “Rapid extraction of bacterial genomic DNA with guanidium thiocyanate”, Lett. Appl. Microbiol., 8, pp 151-156, 1989, Dretzen, G. et al., “A reliable method for the recovery of DNA fragments from agarose and acrylamide gels”, Anal. Biochem., 112, pp 295-298, 1981, WO 94/19454 and Diderichsen et al., “Cloning of aldB, which encodes alpha-acetolactate decarboxylase, an exoenzyme from Bacillus brevis”, J. Bacteriol., 172, pp 4315-4321, 1990.

Procedures for preparing a gene library from an in vitro made synthetic nucleotide source can be found in literature (e.g. described by Stemmer, Proc. Natl. Acad. Sci. USA, 91, pp. 10747-10751, 1994 or WO 95/17413).

The library can also be screened as autonomically replicating plasmid library.

### Manipulating the Nucleic Acid Sequences of a Library

In a particular embodiment the genes of a gene library may before, during or after initiating the screening be subjected to alterations and or mutations by genetic engineering. Generation of libraries of genes encoding variants of enzymes can be done in a variety of ways:

(1) Error prone PCR employs a low fidelity replication step to introduce random point mutations at each round of amplification (Caldwell and Joyce (1992), PCR Methods and Applications vol. 2 (1), pp. 28-33). Error-prone PCR mutagenesis is performed using a plasmid encoding the wild-type, i.e. wt, gene of interest as template to amplify this gene with flanking primers under PCR conditions where increased error rates leads to introduction of random point mutations. The PCR conditions utilized are typically: 10 mM Tris-HCl, pH 8.3, 50 mM KCl, 4 mM MgCl₂, 0.3 mM MnCl₂, 0.1 mM dGTP/dATP, 0.5 mM dTTP/dCTP, and 2.5 u Taq polymerase per 100 micro L of reaction. The resultant PCR fragment is purified on a gel and cloned using standard molecular biology techniques.

(2) Oligonucleotide directed mutagenesis in single codon position (including deletions or insertions), e.g. by SOE-PCR is described by Kirchhoff and Desrosiers, PCR Methods and Applications, 1993, 2, 301-304. This method is performed as follows: Two independent PCR reactions are performed with 2 internal, overlapping primers, wherein one or both contain a mutant sequence and 2 external primers, which may encode restriction sites, thereby creating 2 overlapping PCR fragments. These PCR fragments are purified, diluted, and mixed in molar ratio 1:1. The full length PCR product is subsequently obtained by PCR amplification with the external primers. The PCR fragment is purified on gel and cloned using standard molecular biology techniques.

(3) Oligonucleotide directed randomization in single codon position, such as saturation mutagenesis, may be done e.g. by SOE-PCR as described above, but using primers with randomized nucleotides. For example NN (G/T), wherein N is any of the 4 bases G,A,T or C, will yield a mixture of codons encoding all possible amino acids.

(4) Combinatorial site-directed mutagenesis libraries may be employed, where several codons can be mutated at once using (2) and (3) above. For multiple sites, several overlapping PCR fragments are assembled simultaneously in a SOE-PCR setup.

(5) Another protocol employs synthetic gene libraries preparation. Wild type, i.e. wt, genes can be assembled from multiple overlapping oligonucleotides (typically 40-100 nucleotides in length; (Stemmer et al., (1995), Gene 164, 49-53). By including mixtures of wt and mutant variants of the same oligo at various positions in the gene, the resulting assembled gene will contain mutations at various positions with mutagenic rates corresponding to the ratios of wt to mutant primers.

(6) Still another method employs multiple mutagenic primers to generate libraries with multiple mutated positions. First an uracil-containing nucleotide template encoding a polypeptide of interest is generated and 2-50 mutagenic primers corresponding to at least one region of identity in the nucleotide template are synthezised so that each mutagenic primer comprises at least one substitution of the template sequence (or: insertion/deletion of bases) resulting in at least one amino acid substitution (or insertion/deletion) of the amino acid sequence encoded by the uracil-containing nucleotide template. The mutagenic primers are then contacted with the uracil-containing nucleotide template under conditions wherein a mutagenic primer anneals to the template sequence. This is followed by extension of the primer(s) catalyzed by a polymerase to generate a mixture of mutagenized polynucleotides and uracil-containing templates. Finally, a host cell is transformed with the polynucleotide and template mixture wherein the template is degraded and the mutagenized polynucleotide replicated, generating a library of polynucleotide variants of the gene of interest.

(7) Libraries may be created by shuffling e.g. by recombination of two or more wt genes or genes encoding variant proteins created by any combination of methods (1)-(6) (above) by DNA shuffling.

In the present invention, the nucleic acid sequence may be introduced into the host cell in the form of a nucleic acid construct.

### Expression Vectors

The present invention also relates to recombinant expression vectors comprising the nucleic acid construct of the invention. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome.

The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers which confer antibiotic resistance such as ampicillin, kanamycin, chloramphenicol or tetracycline resistance.

The vectors of the present invention preferably contain an element(s) that permits stable integration of the vector into the host cell's genome or autonomous replication of the vector in the cell independent of the genome.

For integration into the host cell genome, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75: 1433).

More than one copy of a nucleotide sequence of the present invention may be inserted into the host cell to increase production of the gene product. An increase in the copy number of the nucleotide sequence can be obtained by integrating at least one additional copy of the sequence into the host cell genome or by including an amplifiable selectable marker gene with the nucleotide sequence where cells containing amplified copies of the selectable marker gene, and thereby additional copies of the nucleotide sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cell

The present invention further relates to a recombinant host cell comprising the nucleic acid construct or the recombinant expression vector.

In a preferred embodiment, the recombinant host cell is selected from a group comprising bacterial, fungal, and plant cells; more preferably the recombinant host cell is a bacterium, even more preferably it is a Gram-positive bacterium, more preferably it is a prokaryotic cell and most preferably it is a Bacillus cell.

Cells which are able to sporulate are all applicable in the present invention. Useful cells are bacterial cells, preferably Bacillus cells. Preferably, the Bacillus species is selected from the group comprising Bacillus alkalophilus, Bacillus amyloliquefaciens, Bacillus brevis, Bacillus circulans, Bacillus clausii, Bacillus coagulans, Bacillus halodurans, Bacillus lautus, Bacillus lentus, Bacillus licheniformis, Bacillus megaterium, Bacillus pumilus, Bacillus stearothermophilus, Bacillus subtilis, and Bacillus thuringiensis. More preferably, the Bacillus species is Bacillus clausii, Bacillus lentus, Bacillus licheniformis, or Bacillus subtilis. Most preferably, the Bacillus species is Bacillus subtilis or Bacillus licheniformis. Strains of these species are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

### Transformation

The introduction of a vector into a bacterial host cell may, for instance, be effected by protoplast transformation (see, e.g., Chang and Cohen, 1979, Molecular General Genetics 168: 111-115), using competent cells (see, e.g., Young and Spizizin, 1961, Journal of Bacteriology 81: 823-829, or Dubnau and Davidoff-Abelson, 1971, Journal of Molecular Biology 56: 209-221), electroporation (see, e.g., Shigekawa and Dower, 1988, Biotechniques 6: 742-751), or conjugation (see, e.g., Koehler and Thorne, 1987, Journal of Bacteriology 169: 5771-5278).

### Enzymes

In a preferable embodiment of the present invention, the recombinant host cell further contains a gene encoding an enzyme. The gene may be obtained from any prokaryotic, eukaryotic, or other source. The enzyme may be homologous or heterologous to a host cell.

In a more preferred aspect, the enzyme is an oxidoreductase, transferase, hydrolase, lyase, isomerase, or ligase. In an even more preferred aspect, the enzyme is an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, invertase, laccase, another lipase, mannosidase, mutanase, oxidase, pectinolytic enzyme, peroxidase, phytase, polyphenoloxidase, proteolytic enzyme, ribonuclease, transglutaminase or xylanase. More specifically, the enzymes may be the following.

### Parent Proteases

Parent proteases (i.e. enzymes classified under the Enzyme Classification number E.C. 3.4 in accordance with the Recommendations (1992) of the International Union of Biochemistry and Molecular Biology (IUBMB)) include proteases within this group.

Examples include proteases selected from those classified under the Enzyme Classification (E.C.) numbers:

3.4.11 (i.e. so-called aminopeptidases), including 3.4.11.5 (Prolyl aminopeptidase), 3.4.11.9 (X-pro aminopeptidase), 3.4.11.10 (Bacterial leucyl aminopeptidase), 3.4.11.12 (Thermophilic aminopeptidase), 3.4.11.15 (Lysyl aminopeptidase), 3.4.11.17 (Tryptophanyl aminopeptidase), 3.4.11.18 (Methionyl aminopeptidase).

3.4.21 (i.e. so-called serine endopeptidases), including 3.4.21.1 (Chymotrypsin), 3.4.21.4 (Trypsin), 3.4.21.25 (Cucumisin), 3.4.21.32 (Brachyurin), 3.4.21.48 (Cerevisin) and 3.4.21.62 (Subtilisin); 3.4.22 (i.e. so-called cysteine endopeptidases), including 3.4.22.2 (Papain), 3.4.22.3 (Ficain).

3.4.22.6 (Chymopapain), 3.4.22.7 (Asclepain), 3.4.22.14 (Actimidain), 3.4.22.30 (Caricain) and 3.4.22.31 (Ananain);

3.4.23 (i.e. so-called aspartic endopeptidases), including 3.4.23.1 (Pepsin A), 3.4.23.18 (Aspergillopepsin I), 3.4.23.20 (Penicillopepsin) and 3.4.23.25 (Saccharopepsin); and

3.4.24 (i.e. so-called metalloendopeptidases), including 3.4.24.28 (Bacillolysin).

Examples of relevant subtilisins comprise subtilisin BPN', subtilisin amylosacchariticus, subtilisin 168, subtilisin mesentericopeptidase, subtilisin Carlsberg, subtilisin DY, subtilisin 309, subtilisin 147, thermitase, aqualysin, Bacillus PB92 protease, proteinase K, Protease TW7, and Protease TW3.

Specific examples of such readily available commercial proteases include Esperase®, Alcalase®, Neutrase®, Dyrazym®, Savinase®, Pyrase®, Pancreatic Trypsin NOVO (PTN), Bio-Feed® Pro, Clear-Lens Pro® (all enzymes available from Novozymes A/S).

Examples of other commercial proteases include Maxtase®, Maxacal®, Maxapem® marketed by Gist-Brocades N.V., Opticlean® marketed by Solvay et Cie. and Purafect® marketed by Genencor International.

It is to be understood that also protease variants are contemplated as the parent protease. Examples of such protease variants are disclosed in EP 130.756 (Genentech), EP 214.435 (Henkel), WO 87/04461 (Amgen), WO 87/05050 (Genex), EP 251.446 (Genencor), EP 260.105 (Genencor), Thomas et al., (1985), Nature. 318, p. 375-376, Thomas et al., (1987), J. Mol. Biol., 193, pp. 803-813, Russel et al., (1987), Nature, 328, p. 496-500, WO 88/08028 (Genex), WO 88/08033 (Amgen), WO 89/06279 (Novo Nordisk A/S), WO 91/00345 (Novo Nordisk A/S), EP 525 610 (Solvay) and WO 94/02618 (Gist-Brocades N.V.).

### Parent Lipases

Parent lipases (i.e. enzymes classified under the Enzyme Classification number E.C. 3.1.1 (Carboxylic Ester Hydrolases) in accordance with the Recommendations (1992) of the International Union of Biochemistry and Molecular Biology (IUBMB)) include lipases within this group.

Examples include lipases selected from those classified under the Enzyme Classification (E.C.) numbers:

3.1.1 (i.e. so-called Carboxylic Ester Hydrolases), including (3.1.1.3) Triacylglycerol lipases, (3.1.1.4.) Phosphorlipase A2.

Examples of lipases include lipases derived from the following microorganisms Humicola, e.g. H. brevispora, H. lanuginosa, H. brevis var. thermoidea and H. insolens (U.S. Pat. No. 4,810,414). Pseudomonas, e.g. Ps. fragi, Ps. stutzeri, Ps. cepacia and Ps. fluorescens (VVO 89/04361), or Ps. plantarii or Ps. gladioli (U.S. Pat. No. 4,950,417 (Solvay enzymes)) or Ps. alcaligenes and Ps. pseudoalcaligenes (EP 218 272) or Ps. mendocina (VVO 88/09367; U.S. Pat. No. 5,389,536). Fusarium, e.g. F. oxysporum (EP 130,064) or F. solani pisi (WO 90/09446). Mucor (also called Rhizomucor), e.g. M. miehei (EP 238 023). Chromobacterium (especially C. viscosum). Aspergillus (especially A. niger). Candida, e.g. C. cylindracea (also called C. rugosa) or C. antarctica (VVO 88/02775) or C. antarctica lipase A or B (WO 94/01541 and WO 89/02916). Geotricum, e.g. G. candidum (Schimada et al., (1989), J. Biochem., 106, 383-388). Penicillium, e.g. P. camembertii (Yamaguchi et al., (1991), Gene 103, 61-67). Rhizopus, e.g. R. delemar (Hass et al., (1991), Gene 109, 107-113) or R. niveus (Kugimiya et al., (1992) Biosci.Biotech. Biochem 56, 716-719) or R. oryzae. Bacillus, e.g. B. subtilis (Dartois et al., (1993) Biochemica et Biophysica acta 1131, 253-260) or B. stearothermophilus (JP 64/7744992) or B. pumilus (WO 91/16422).

Specific examples of readily available commercial lipases include Lipolase®, Lipolase®Ultra, Lipozyme®, Palatase®, Novozym® 435, Lecitase® (all available from Novozymes A/S). Examples of other lipases are Lumafast®, Ps. mendocian lipase from Genencor Int. Inc.; Lipomax®, Ps. pseudoalcaligenes lipase from Gist Brocades/Genencor Int. Inc.; Fusarium solani lipase (cutinase) from Unilever; Bacillus sp. lipase from Solvay enzymes. Other lipases are available from other companies.

It is to be understood that also lipase variants are contemplated as the parent enzyme. Examples of such are described in e.g. WO 93/01285 and WO 95/22615.

### Parent Oxidoreductases

Parent oxidoreductases (i.e. enzymes classified under the Enzyme Classification number E.C. 1 (Oxidoreductases) in accordance with the Recommendations (1992) of the International Union of Biochemistry and Molecular Biology (IUBMB)) include oxidoreductases within this group.

Examples include oxidoreductases selected from those classified under the Enzyme Classification (E.C.) numbers:

Glycerol-3-phosphate dehydrogenase _NAD+_ (1.1.1.8), Glycerol-3-phosphate dehydrogenase _NAD(P)+_ (1.1.1.94), Glycerol-3-phosphate 1-dehydrogenase _NADP_ (1.1.1.94), Glucose oxidase (1.1.3.4), Hexose oxidase (1.1.3.5), Catechol oxidase (1.1.3.14), Bilirubin oxidase (1.3.3.5), Alanine dehydrogenase (1.4.1.1), Glutamate dehydrogenase (1.4.1.2), Glutamate dehydrogenase _NAD(P)+_ (1.4.1.3), Glutamate dehydrogenase _NADP+_ (1.4.1.4), L-Amino acid dehydrogenase (1.4.1.5), Serine dehydrogenase (1.4.1.7), Valine dehydrogenase _NADP+_ (1.4.1.8), Leucine dehydrogenase (1.4.1.9), Glycine dehydrogenase (1.4.1.10), L-Amino-acid oxidase (1.4.3.2.), D-Amino-acid oxidase(1.4.3.3), L-Glutamate oxidase (1.4.3.11), Protein-lysine 6-oxidase (1.4.3.13), L-lysine oxidase (1.4.3.14), L-Aspartate oxidase (1.4.3.16), D-amino-acid dehydrogenase (1.4.99.1), Protein disulfide reductase (1.6.4.4), Thioredoxin reductase (1.6.4.5), Protein disulfide reductase (glutathione) (1.8.4.2), Laccase (1.10.3.2), Catalase (1.11.1.6), Peroxidase (1.11.1.7), Lipoxygenase (1.13.11.12), Superoxide dismutase (1.15.1.1)

Said Glucose oxidases may be derived from Aspergillus niger. Said Laccases may be derived from Polyporus pinsitus, Myceliophtora thermophila, Coprinus cinereus, Rhizoctonia solani, Rhizoctonia praticola, Scytalidium thermophilum and Rhus vernicifera. Bilirubin oxidases may be derived from Myrothechecium verrucaria. The Peroxidase may be derived from e.g. Soy bean, Horseradish or Coprinus cinereus. The Protein Disulfide reductases Protein Disulfide reductases of bovine origin, Protein Disulfide reductases derived from Aspergillus oryzae or Aspergillus niger, and DsbA or DsbC derived from Escherichia coli.

Specific examples of readily available commercial oxidoreductases include Gluzyme (enzyme available from Novozymes A/S). However, other oxidoreductases are available from others.

It is to be understood that also variants of oxidoreductases are contemplated as the parent enzyme.

### Parent Carbohydrases

Parent carbohydrases may be defined as all enzymes capable of breaking down carbohydrate chains (e.g. starches) of especially five and six member ring structures (i.e. enzymes classified under the Enzyme Classification number E.C. 3.2 (glycosidases) in accordance with the Recommendations (1992) of the International Union of Biochemistry and Molecular Biology (IUBMB)).

Examples include carbohydrases selected from those classified under the Enzyme Classification (E.C.) numbers:

alfa-amylase (3.2.1.1) alfa-amylase (3.2.1.2), glucan 1,4-alfa-glucosidase (3.2.1.3), cellulase (3.2.1.4), endo-1,3(4)-beta-glucanase (3.2.1.6), endo-1,4-beta-xylanase (3.2.1.8), dextranase (3.2.1.11), chitinase (3.2.1.14), polygalacturonase (3.2.1.15), lysozyme (3.2.1.17), beta-glucosidase (3.2.1.21), alfa-galactosidase (3.2.1.22), beta-galactosidase (3.2.1.23), amylo-1,6-glucosidase (3.2.1.33), xylan 1,4-beta-xylosidase (3.2.1.37), glucan endo-1,3-beta-D-glucosidase (3.2.1.39), alfa-dextrin endo-1,6-glucosidase (3.2.1.41), sucrose alfa-glucosidase (3.2.1.48), glucan endo-1,3-alfa-glucosidase (3.2.1.59), glucan 1,4-beta-glucosidase (3.2.1.74), glucan endo-1,6-beta-glucosidase (3.2.1.75), arabinan endo-1,5-alfa-arabinosidase (3.2.1.99), lactase (3.2.1.108), and chitonanase (3.2.1.132).

Specific examples of readily available commercial carbohydrases include Alpha-Gale, Bio-Feed® Alpha, Bio-Feed® Beta, Bio-Feed® Plus, Bio-Feed® Plus, Novozyme® 188, Carezyme®, Celluclast®, Cellusoft®, Ceremyl®, Citrozym®, Denimax®, Dezyme®, Dextrozyme®, Finizym®, Fungamyl®, Gamanase®, Glucanex®, Lactozym®, Maltogenase®, Pentopan®, Pectinex®, Promozyme®, Pulpzyme®, Novamyl®, Termamyl®, AMG (Amyloglucosidase Novo), Maltogenase®, Aquazym®, Natalase® (all enzymes available from Novozymes A/S). Other carbohydrases are available from other companies.

It is to be understood that also carbohydrase variants are contemplated as the parent enzyme.

The activity of the above enzymes can be determined as described in “Methods of Enzymatic Analysis”, third edition, 1984, Verlag Chemie, Weinheim, vol. 3.

### EXAMPLES

### Example 1

### Recombinant Host with Sporulation Under Phosphate Starvation Control

### Bacterial Strains and Medium

A derivative of Bacillus subtilis 168 (F. Kunst, et al. The complete genome sequence of the gram-positive bacterium Bacillus subtilis. Nature 390(6657):249-256 (1997)), denoted MIBG601, was used in this study.

Schaeffer's sporulation medium (SSM or SM) is a commonly used general purpose sporulation medium for Bacillus subtilis and related species. The composition of Schaeffer's medium (SSM) used herein was as follows:

| Ingredients | Supplier and number | Amount | |
|---|---|---|---|
| Peptone Bacto | Difco 211820 or 211677 | 5 | g |
| Beef extract | Merck 105886 | 0.25 | g |
| KCl | Merck 104936 | 1 | g |
| MnSO₄ 1% | MSA-SUB-RE-5022 | 0.15 | ml |
| FeSO₄ 1% | MSA-SUB-RE-5023 | 0.03 | ml |
| Ca(NO₃)₂•4H₂O | Merck 102121 | 0.24 | g |
| Agar | Difco/Oxoid 102-3799 | 20 | g |
| Water | | filled up to 1000 | ml |
| pH 7.0-7.4 | | | |

### DNA Manipulations

B. subtilis transformations were performed as described previously (Anagnostopolous, C., and J. Spizizen. 1961. Requirements for transformation in Bacillus subtilis. J. Bacteriol. 81:741-746). All routine molecular biological procedures were performed according to the protocols described by Sambrook et al. (1989).

### Expression Constructs

Expression constructs were made in either a plasmid or in a linear integration vector. In both ways the final gene construct is integrated on the Bacillus chromosome by homologous recombination into the AmyE locus. Cloning in the plasmid was done according to the protocols described by Sambrook et al. (1989). The linear integration vector is a PCR fusion product made by fusion of the gene of interest between two Bacillus subtils homologous chromosomal regions along with a low-phosphate inducible promoter and a spectinomycin resistance marker. The fusion is made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68).

At first, 8 fragments were PCR amplified:
A) The spectinomycin gene spec^{R} was amplified from S. aureus (EMBL: AP009324, issued on 10 Sep. 2007) by primers p3 (SEQ ID NO: 3)+p4 (SEQ ID NO: 4) leading to a 1.2 kb fragment;
B) a segment of the pAM-beta plasmid (EMBL: GU128949) was amplified by primer p5 (SEQ ID NO: 5) and p6 (SEQ ID NO: 6) leading to a 0.2 kb fragment;
C) the Ppst promoter region was amplified from B. licheniformis (EMBL: AE017333, issued: 21-SEP-2004) by primer p7 (SEQ ID NO: 7) and p8 (SEQ ID NO: 8) leading to a 0.4 kb fragment;
D) the ribosomal binding site of B. clausii (EMBL: A22550, issued: 01-JUN-1995) was hooked up to the sigF sequence by prolonging the p8 and p9 primers;
E) the sigF gene of B. subtilis 168 was ampified by primer p9 (SEQ ID NO: 9) and p10 (SEQ ID NO: 10) leading to a 0.8 kb fragment;
F) the terminator region aprH from B. clausii (EMBL: A22550, issued: 1 Jun. 1995) was amplified by primer p11 (SEQ ID NO: 11) and p12 (SEQ ID NO: 12) leading to a 0.2 kb fragment;
G) the upstream flanking region from the amyE region of B. subtilis 168 was amplified by primer p1 (SEQ ID NO: 1) and p2 (SEQ ID NO: 2) leading to a 2.2 kb fragment;
H) the downstream flanking region from the amyE region of B. subtilis 168 was amplified by primer p13 (SEQ ID NO: 13) and p14 leading to a 2.7 kb fragment.

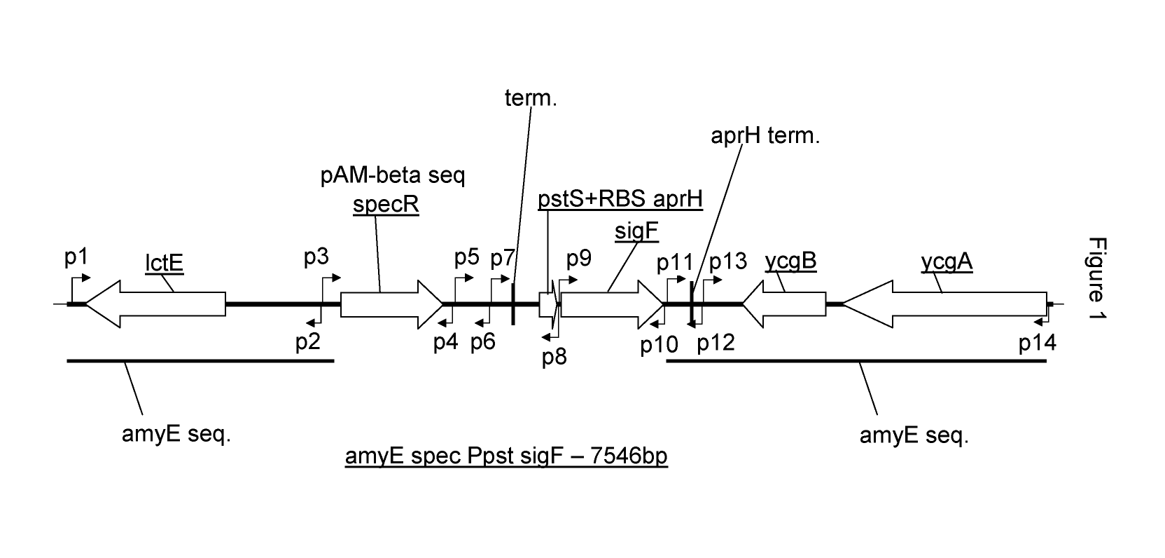

The obtained fragments were then fused together by SOE PCR:
I) The PCR fragments in A), B) and C) were assembled by primer p3 (SEQ ID NO: 3) and p8 (SEQ ID NO: 8) leading to a 1.8 kb fragment;
J) the PCR fragments in D), E) and F) were assembled by primer p9 (SEQ ID NO: 9) and p12 (SEQ ID NO: 12) leading to a 1.0 kb fragment;
K) the fragments in I) and J) were assembled by primer p3 (SEQ ID NO: 3) and p12 (SEQ ID NO: 12) leading to a 2.8 kb fragment; and,
L) finally, the flanking regions in G) and H) as well as the fragment in K) were assembled by primer p1 (SEQ ID NO: 1) and p14 (SEQ ID NO: 14) leading to a 5.7 kb fragment (SEQ ID NO: 15). The obtained amyE::spec^{R} Ppst sigF fragment is shown in FIG. 1.

### Strain Construction

The above amyE::spec^{R} Ppst sigF PCR fragment was transformed to a competent B. subtilis 168 and selected for resistance towards spectinomycin. After selection, a transformed strain B. subtilis 168 amyE::spec^{R} Ppst sigF (referred to as PP2941 herein) was obtained. The chromosomal insert amyE::spec^{R} Ppst sigF was confirmed by DNA sequencing.

### Example 2

### Phosphate Starvation-Sporulation Leads to Survival at pH 4 and 80° C.

This example shows that the sporulation can be induced on the right medium and spores can survive under an agar layer with pH 4 and incubation at high temperature. A B. subtilis A168 derivate was used as cloning and screening host, BE158 is identical except it carries an additional alpha-amylase gene, PP510 is the B. subtilis A164 wild type strain, PP2941 is BE158 with the phosphate starvation inducible sporulation.

### Material and Methods

Schaeffer's medium was used undiluted or 2, 5 and 10-fold diluted with 20 g/l Agar. Luria Bertani (LB) medium was used undiluted or 2, 5 and 10-fold diluted with 20 g/l Agar.

Glycerol stocks were prepared from an overnight culture diluted with medium to 0.002 OD at 600 nm. 60 ul of 200 fold diluted glycerol stocks of the following strains BE158, PP510, and PP2941 were plated onto 9 cm Petri plates containing 15 ml of one of the prepared agar mixes. The plates were incubated for 18 hours at 32° C. Sporulation was first checked by microscopy. Then a 16 ml agar overlay with 100 mM acetate buffer at pH 4 was casted on top and the plates were incubated 30 min at 80° C. in a waterbath or at 80° C. in an incubator. Ten colonies were picked, striked on new plates and incubated over night to check survival.

### Results

On undiluted LB and diluted LB agar, the natural sporulating strain PP510 and the phosphate starvation induced sporulating strain PP2941 did not sporulate, because no spores were seen by microscopy even after 4 days of incubation.

On Schaeffers and diluted Schaeffers agar no spores were observed after 19.5 hours at at 32° C. After further one day incubation at 37° C. the following observation was made:

### PP510:

The colonies were smaller on 5× and 10× diluted Schaeffer's medium than on undiluted Schaeffer's medium.
1× diluted Schaeffer's medium: spores present, about ⅓ of Bacillus length
5× diluted Schaeffer's medium: spores present, also in cells
10× diluted Schaeffer's medium lots of spores, mostly in cells

### PP2941:

The colonies were less dense with higher dilution of Schaeffers agar
1× diluted Schaeffers medium: no spores
5× diluted Schaeffers medium: about one spore of 50 cells
10× diluted Schaeffers medium: about one spore of 50 cells
Several colonies were picked from these plates after two days growth at 37° C.; table 1 shows the number of colonies grown overnight at 37° C. of total colonies picked.

TABLE 1

| | 10x diluted | 5x diluted | |
|---|---|---|---|
| | Schaeffer's | Schaeffer's | |
| Strain | medium | medium | result |
| MIBG601 | 0 of 10 | 0 of 10 | No sporulation - no survival |
| PP510 | 10 of 10 | 3 of 4 | Microscopically checked |
| | | | that grown colonies were |
| | | | Bacillus |
| PP2941 | 8 of 8 | 10 of 10 | Microscopically checked |
| | | | that grown colonies were |
| | | | Bacillus |

### Conclusion

Sporulation and survival after overlay with pH4 and incubation for 30 min at 80° C. in a waterbath was found for the phosphate starvation induced sporulating strains only on 5 and 10× diluted Schaeffer's medium. A usual sporulating strain also sporulated on undiluted Schaeffer's medium. This shows that the induction by phosphate starvation worked. Regrowth after picking was near 100%.

### Example 3

### Best Conditions for Sporulation and Survival of Strain PP2941

### Materials and Methods

80 ul of a 200× dilution of the 2 mOD glycerol stocks were plated out on different plates (Table 2).

TABLE 2

| | BE158 | PP2941 |
|---|---|---|
| LB | | X |
| LB Kan 10 + Spec 10 | | X |
| Schaeffer's not diluted | x | X |
| Schaeffer's 5x diluted | x | X |
| Schaeffer's 10x diluted | x | X |
| Schaeffer's 20x diluted | x | X |
| Schaeffer's 50x diluted | x | X |

Incubation overnight at 37° C.

When spores were present microscopically, an overlay with 15 g/l agar and 1 g/l red starch (Megazyme, Ireland) was casted. In case of MIBG601 with BE158, 100 mM acetate buffer with pH 5.8 were present in the overlay. In case of PP2941, 100 mM acetate buffer with pH 4 were present in the overlay. BE158 was incubated thereafter at room temperature in order to check expression of the amylase. PP2941 was treated 30 min 80° C. in a waterbath and survival was checked by picking colonies onto a fresh plate.

### Results:

After 18 and 24 hour incubation at 37° C., microscopy of PP2941 grown on LB-Kan10-Spec10, Schaeffer's agar 50×, 20×, 10× and 5× diluted showed that no spores were present. Incubation was continued at 37° C.

After 41 hours growth at 37° C., microscopy was repeated with the following result:

| Medium | observation |
|---|---|
| LB | no spores, thick rods |
| SM 50x | no spores, often shorter cells |
| SM 20x | spores |
| SM 10x | spores |
| SM 5x | many spores |
| SM undiluted | no spores |

The overlay with red starch at pH 4 was cast onto PP2941 and the plates were incubated 30 min at 80° C. in a waterbath. Then colonies were picked on new plates and grown overnight. Grown colonies showed survival after the low pH and heat treatment. Picking of overlaid colonies from spore producing strains resulted in (Table 3):

TABLE 3
Growth of PP2941 on medium with different dilution.

| | | Grown/ | |
|---|---|---|---|
| Strain | Medium | picked | Survival (%) |
| PP2941 | Schaeffer's undiluted | 0/6 | 0 |
| PP2941 | Schaeffer's 5x diluted | 6/6 | 100 |
| PP2941 | Schaeffer's 10x diluted | 7/7 | 100 |
| PP2941 | Schaeffer's 20x diluted | 6/7 | 86 |
| PP2941 | Schaeffer's 50x diluted | 7/8 | 88 |
| PP2941 | LB | 0/6 | 0 |
| PP2941 | LB-KAN10-Spec10 | 0/6 | 0 |
| Picked with metal | Schaeffer's 5x diluted | 6/6 | 100 |
| pin from colony | | | |
| picker | | | |
| Picked with metal | Schaeffer's 10x diluted | 5/5 | 100 |
| pin from colony | | | |
| picker | | | |

Picking of spores also worked well with the metal pin from the colony picker. PP2941 survived best with diluted Schaeffer's medium. The growth was optimal after 5× to 10× dilution. Plates with BE158 were incubated after 41 hours growth at 37° C. with a red starch overlay at pH 5.8 and incubated at room temperature overnight; all colonies had large clearing zones the next day. The results (Table 4) show clearly that amylase was produced on the medium also supporting spore formation.

TABLE 4

| | Clearing zone | |
|---|---|---|
| Medium | radius (mm) | Clearing strength |
| Schaeffer's 50x diluted | 5 | Clear |
| Schaeffer's 5x diluted | 10 | Clear |
| Schaeffer's undiluted | 10 | Contrast difficult due to |
| | | brownish colour of Schaeffer's |
| | | medium |

Further experiments showed that the non-sporulating strain MIBG601 survived at RT under a pH 5.8 overlay, but was killed at pH 4 and 3.8. With an overlay at pH 5.8, it was also killed after 30 min at 80° C. in a waterbath. The strain PP2941 with the phosphate starvation induced sporulation survived all of these conditions after growth for 2 days at 37° C. on 5-fold diluted Schaeffer's medium. On 2-fold diluted Schaeffer's medium the survival rate was still 75% of the colonies.

### Conclusion

5× diluted Schaeffer's medium was the best choice for supporting sporulation and amylase expression. After 1 day growth at 37° C. the colonies had a diameter of about 1 mm. After 2 days spores were visible and survived the redstarch overlay at pH 4 and incubation at 80° C. in the waterbath for 30 min. These colonies can also be picked up with a steel needle. The amylase was shown to give a 10 mm clearing zone after overnight RT incubation with the pH 5.8 red starch overlay with the parent strain MIBG601 containing BE158.

### Example 4

### Screening of Protease Using Recombinant Strain

Genomic DNA was made from a Bacillus subtilis strain containing a Nocardiopsis S2A protease, the so-called “10R protease”, under control of a strong promoter, fused to a Savinase signal peptide (as shown, e.g., in WO 05/123914) and integrated with a chloramphenicol resistance gene into the pel locus of the Bacillus strain. The chromosomal DNA was transformed for integration into B. subtilis MIBg601 as a non-sporulating strain) and PP2941 (sporulating strain) was transformed with SOL000 genomic DNA, positive transformants were selected via the chloramphenicol resistance. Colonies with clearing zones were selected and one single colony from MiBG601 and from PP2941 were used for the following tests.

The strains were inoculated into Schaffer's bouillon (same as Schaeffer's medium without addition of agar), which was diluted lx, 2×, 3×, 5×, and 10× and CaCl₂ was added to a final concentration of 2.8 mM. The cultures grew for several days at 30° C. 220 rpm and checked microscopically for sporulation. After 2 days sporulation was seen in PP2941-SOL000 and spore concentration increased at day 3 and day 4. There was no difference between the dilutions, so that phosphate levels in an undiluted Schaeffer's boullion was already getting limited during growth. This was due to higher cell concentrations than obtained on a solid medium. MiBg601-SOL000 did not sporulate under any of these conditions.

Each day supernatant samples were taken and analyzed for protease activity in order to determine how much protease was produced. The EnzChek Protease Assay Kit (Invitrogen) with red Fluorescence and the substrate was diluted to a final concentration of 1 mg/ml with 0.1M NaHCO₃ pH=8.0. The obtained fluorescence was compared to a standard curve of the 10R protease. In undiluted Schaeffer's bouillion both strains obtained similar protease concentration with 35 ppm after 2 days, 25 ppm after 3 days and 30 ppm after 4 days. Similar protease concentration was obtained by cultivation in TB-Glycerol medium.

Culture samples of MIBG601-SOL000 and PP2941-SOL000 after 4 days incubation were tested for spore survival. Therefore, for the control the culture was diluted with Schaeffer's media: 3×, 10×, 50×, 100× and 200×, 60 μl were plated out on LB cam+skim milk, and incubated at RT for 4 days. In order to determine the number of spores, a heat treatment of the 500 ul culture broth was applied by incubating it at 70° C. for 30 min. These were diluted and plated out the same way as done for the control. In order to determine the spore survival in detergent, 885 ul culture broth with 8×10⁸ cells/ml and 100 ul 12.3 g/l detergent and 13 ul water with 1000° dH were mixed. For a second setup a higher detergent concentration of 44 g/l was used. These were incubated for 6 hours at room temperature and diluted 5-, 10-, 50-, and 100-fold. 50 μl were plated out on LB cam+skim milk and incubated at RT for 4 days. Results after the above heat treatment and detergent treatment were shown in Table 5 and 6, respectively.

TABLE 5
Heat treatment.

| | |
|---|---|
| MiBg601-10R | PP2941-10R-4 |

| Dilution | −HEAT | +HEAT | −HEAT | +HEAT |
|---|---|---|---|---|
| 1x | TMTC | 0 | TMTC | TMTC |
| 3x | TMTC | 0 | TMTC | TMTC |
| 10x | 0* | 0 | TMTC | TMTC |
| 50x | 0* | 1 | TMTC | 6-8000 |
| 100x | 0* | 0 | App. 8000 | App. 4000 |
| 200x | 0* | 0 | App. 6000 | App. 2000 |

TMTC = Too many to count = more than 8,000 colonies.
*= the dilution was made by taking out from the former dilution.

TABLE 6
Detergent treatment.

| | |
|---|---|
| 1.23 g/l | 4.4 g/l |

| | MiBg601- | PP2941- | MiBg601- | PP2941- |
|---|---|---|---|---|
| Dilution | 10R | 10R-4 | 10R | 10R-4 |
| 5x | 17 | TMTC | 2 | TMTC |
| 10x | 2 | TMTC | 6 | TMTC |
| 50x | 3 | TMTC | 2 | TMTC |
| 100x | 1 | app.3000 | 1 | App.3000 |

These results show that PP2941 was sporulating, the spores can withstand heat and detergent and the growth conditions allow sufficient protease production for screening purposes.

### Example 5

### Screening of Amylase Under High Temperature and Low pH

After showing that PP2941 can survive under harsh conditions, a screening setup was established for screening an amylase at high temperature and low pH. BE158 was taken as a wild type amylase. BE1093 was earlier found as an improved variant under such conditions. Here, relevant conditions were found to distinguish the better variant from the wild type directly on plates.

### Materials and Methods

A promoter operably linked to an amylase gene, everything flanked by DNA regions identical to the genomic pectate lyase gene (PEL) of the host was transformed into B. subtilis PP2941 to achieve integration by homologous recombination into PEL. Three different amylase genes were used with BE158 being the wild type and BE1093 being an improved variant. Overnight grown cultures were diluted with growth medium to 0.002 OD at 600 nm and glycerol stocks were prepared.

9 cm Petri dishes were filled with diluted Schaeffer's medium. The agar containing Schaeffer's medium was diluted with 20 g/L agar in deionized water. 2.8 mM CalCl₂ were added to adjust the calcium to 16 degree water hardness. 6 ug/ml Chloramphenicol were added. Schaeffer's medium was diluted 3.3 and 5 fold.

60 ul of glycerol stocks of B. subtilis PP2941 with the BE158 or BE1093 amylase were plated out onto the Petri dishes. Also mixes were prepared and plated out with 90% from a BE158 and 10% from a BE1093 glycerol stock. These plates were grown for 40 hours at 37° C. An overlay agar was prepared with pH 3.8, 3.9 and 4.0 following this scheme e.g. for 150 ml: 75 ml 60° C. warm, autoclaved unbuffered red starch agar with 15 g/l agar and 3 g/l red starch (Megazyme) was mixed with 60° C. warm ingridients such as 60 ml sterile purified water and 15 ml 1M sodium acetate buffer at pH 3.8, 3.9 or 4.0. 6 ug/ml chloramphenicol and 200 ug/ml ampenicillin were added as antibiotics to ensure sterility and selectivity for the amylase gene.

Overlays were casted by adding 16 ml onto the grown colonies ensuring an even spreading of the agar layer. The plates needed 10 min under the flow bench to solidify and were then incubated in a pre-warmed incubator at 75° C. Earlier it was measured that the temperature in the agar adjusted to a final of approximately 70° C. The plates were incubated 22.5 hours prior to analysis of clearing zones and picking of colonies with larger clearing zones from the mixed plate. Picked and regrown colonies were sequenced in order to see, if the improved variant had been picked.

### Results:

After 22.5 hours at 75° C., the plates were analyzed (see Table 7). The number is a measure for the radius of the clearing zone in mm and the letter codes for the strength of the clearing zone with the four degrees very clear (vc), clear (c), weak (w) and very weak (vw):

TABLE 7

| | 3.3-fold diluted | | | 5-fold diluted | | |
|---|---|---|---|---|---|---|
| | Schaeffer's medium | | | Schaeffer's medium | | |
| pH | BE158 | BE1093 | 90/10 | BE158 | BE1093 | 90/10 |
| 3.8 | 1 c | 2 vc | Difference visible, | 1 c | 4 vc | Difference visible, |
| | | | 11 positive of 38 | | | 9 positives of 141 |
| | | | total colonies | | | total colonies |
| 3.9 | 1.5 c | 2 c | Small difference | 0.5 vc | 1.5 vc | Small difference |
| 4.0 | 4 c | 4 c | No difference | 2 c | 5 c | No clear difference |

Positives from pH 3.8 on 5-fold diluted Schaeffer's medium were picked and grown on a new plate. DNA sequencing showed that five out of seven positives had the same improved amylase variant which had the following amino acid substitutions: E129V, K177L and R179E.

1-27. (canceled)

28. A method of screening for an improved enzyme variant, said method comprising the steps of:
(i) providing a recombinant host cell capable of sporulating comprising a polynucleotide encoding a sporulation factor and a polynucleotide encoding an enzyme variant which are operably linked to an inducible promoter;
(ii) culturing the host cell under conditions suitable to induce the formation of spores;
(iii) culturing the spores obtained in step (ii) in a medium containing a substrate for the enzyme variant; and
(iv) determining the activity of the enzyme variant.

29. The method of claim 28, wherein the culturing of the spores is done under a stress condition.

30. The method of claim 29, wherein the stress condition includes acidic pH, high temperature, lack of nutrients, presence of one or more toxin and/or presence of one or more detergent.

31. The method of claim 29, wherein the culturing is performed at a pH value of at most 6.0.

32. The method of claim 29, wherein the culturing is done at a temperature of at least 70° C., 75° C., 80° C., 85° C., or 90° C.

33. The method of claim 28, wherein the formation of spores is induced by culturing the host cell in a sporulation medium.

34. The method of claim 28, wherein the activity of the enzyme variant is determined by an agar overlay assay.

35. The method of claim 28, wherein the enzyme is an oxidoreductase, transferase, hydrolase, lyase, isomerase, or ligase.

36. The method of claim 28, wherein the inducible promoter is induced by phosphorous starvation.

37. The method of claim 28, wherein the recombinant host cell is selected from a group comprising bacterial, fungal, and plant cells.

38. The method of claim 28, wherein the recombinant host cell is a Bacillus cell.

39. A nucleic acid construct comprising a polynucleotide encoding a sporulation factor and a polynucleotide encoding an enzyme variant which are operably linked to a phosphorus starvation inducible promoter.

40. The nucleic acid construct of claim 39, wherein the enzyme is an oxidoreductase, transferase, hydrolase, lyase, isomerase, or ligase.

41. The nucleic acid construct of claim 39, wherein the phosphorus starvation inducible promoter is pstS.

42. The nucleic acid construct of claim 39, further comprising a polynucleotide encoding an antibiotic resistance selectable marker.

43. The nucleic acid construct of claim 42, wherein the selectable marker provides resistance towards ampicillin, kanamycin, tetracycline, chloramphenicol, neomycin or spectinomycin.

44. A recombinant expression vector comprising the nucleic acid construct of claim 39.

45. A recombinant cell comprising the nucleic acid construct of claim 39.

46. The recombinant cell of claim 45, which is a bacterial, fungal or plant cell.

47. The recombinant cell of claim 46, which is a Bacillus cell selected from the group comprising Bacillus alkalophilus, Bacillus amyloliquefaciens, Bacillus brevis, Bacillus circulans, Bacillus clausii, Bacillus coagulans, Bacillus halodurans, Bacillus lautus, Bacillus lentus, Bacillus licheniformis, Bacillus megaterium, Bacillus pumilus, Bacillus stearothermophilus, Bacillus subtilis, and Bacillus thuringiensis.
